(19) United States
(12) Patent Application Publication
(10) Pub. No.: US 2016/0106868 A1
(43) Pub. Date: Apr. 21, 2016

(54) OXYGEN FREE RADICAL DETECTION

(71) Applicant: Progenitec Inc., Arlington, TX (US)

(72) Inventors: WENJING HU, Arlington, TX (US); LIPING TANG, Arlington, TX (US)

(21) Appl. No.: 14/515,475

(22) Filed: Oct. 15, 2014

(51) Int. Cl. A61K 49/00

(52) U.S. Cl. CPC A61K 49/0093; A61K 49/0032; A61K 49/0054

(57) ABSTRACT

Ratiometric techniques can be used to determine activity levels of reactive oxidation species (“ROS”) in a target area (in vitro or in vivo) to indicate inflammatory responses and/or infection. Target areas may be a tissue surface, a wound, and/or a body cavity. A ROS-sensitive bioluminescent agent and ROS-insensitive fluorescent reference dye are conjugated to surfaces, biocompatible particles, or polymer carriers. The compounds may be constituted as a spray, solution for injection, a wound dressing, medical implants, and/or medical device. The bioluminescent compound and reference fluorescent intensity ratios can be calculated to reflect the extent of localized ROS activities.

### BACKGROUND

1. Technical Field

The present disclosure relates generally to detection of inflammatory responses and/or infection. In particular, the present disclosure relates to assessment of the extent of inflammatory responses and/or infection in vitro and/or in vivo by detecting the presence of reactive oxygen species.

2. Description of Related Art

Tissue inflammation may generally be indicative of a variety of injuries and/or diseases, including but not limited to gunshot or other wounds, burns, heart attacks, arthritis, asthma, and cancer. Severe inflammatory responses can endanger or even kill an affected subject. In addition, acute and chronic inflammation are responsible for a wide variety of inflammatory diseases including atherosclerosis, arthritis, celiac disease, chronic obstructive pulmonary disease, irritable bowel disease, multiple sclerosis, psoriasis, vasculitis, lupus, etc. Typically, inflammatory responses caused by external stimuli (e.g., injury, invasion of foreign bodies, and/or microorganisms) may be identified by observation of five principal symptoms: heat, pain, redness, swelling, and immobility. Conversely, the extent of internal or subcutaneous inflammation and/or infection may not be assessed as readily as externally-caused inflammation and/or infection. Traditionally, histological evaluation of tissue biopsy may provide direct measurements of inflammatory responses. However, a tissue biopsy is a relatively invasive and potentially unreliable method of assessing the overall degree of inflammation. Analyzing white blood cell counts and inflammatory cytokine concentrations in blood may provide some indications of systemic immune reactions. Bacterial culture may routinely be carried out to determine the cause of infection. However, the results from such analyses may not accurately estimate the extent of localized and internal inflammatory responses and/or infection. Therefore, there is a need for the development of new method to assess the extent of inflammatory responses and infection in vivo in clinic practice.

Inflamed tissues are typically filled with a variety of immune cells, including macrophages/monocytes, polymorphonuclear neutrophils (“PMNs”), mast cells, lymphocytes, and dendritic cells that participate in the pathogenesis of inflammatory diseases. Among all immune cells, PMNs may be the most abundant type, arriving in large numbers at the injured tissue site within minutes after trauma or infection. Therefore, histological evaluations of PMN accumulation in the tissue are often carried out to estimate the extent of acute inflammatory response at the inflamed and/or infected site. Activated PMNs often undergo respiratory burst, releasing a variety of reactive oxygen species (“ROS”), including but limited to superoxide, hydrogen peroxide, and hyperchloric acid. Release of ROS may result in oxidative damage to foreign microorganism, injured cells, and even healthy cells. The extent of ROS production in tissue may reflect the degree of inflammatory reactions and/or infection.

Various methods have been developed to assess the release of ROS by inflammatory cells. These methods include spectrophotometrical measurements, electron spin resonance spectroscopy, ELISA, chemiluminescence, etc. These methods have been used extensively to study the extent of ROS production by PMN in vitro. However, most of these methods cannot be used to effectively measure the PMN-associated ROS responses in vivo.

### SUMMARY

In one embodiment, a composition for detecting reactive oxidation species activities is disclosed. The composition has a reactive oxidation species-sensitive chemiluminescent compound and a reactive oxidation species-insensitive fluorescent dye.

In another embodiment, method of detecting reactive oxygen species activities is disclosed. The method includes: applying a compound to a target area, the compound comprising a reactive oxidation species-sensitive chemiluminescent compound and a reactive oxidation species-insensitive fluorescent dye; detecting a first emission intensity of chemiluminescence from the reactive oxidation species-sensitive chemiluminescent compound; detecting a second emission intensity of fluorescence from the reactive oxidation species-insensitive fluorescent dye; and calculating reactive oxygen species activities by comparing the first emission intensity against the second emission intensity.

The present disclosure will now be described more fully with reference to the accompanying drawings, which are intended to be read in conjunction with both this summary, the detailed description, and any preferred or particular embodiments specifically discussed or otherwise disclosed. This disclosure may, however, be embodied in many different forms and should not be construed as limited to the embodiments set forth herein; rather, these embodiments are provided by way of illustration only so that this disclosure will be thorough, and fully convey the full scope of the present disclosure to those skilled in the art.

### BRIEF DESCRIPTION OF THE DRAWINGS

Non-limiting and non-exhaustive embodiments of the present disclosure are described with reference to the following figures, wherein like reference numerals refer to like parts throughout the various views unless otherwise specified.

FIG. 1 depicts a basic design schematic of reactive oxygen species detecting compounds according to embodiments of the present disclosure;

FIG. 2 depicts various applications of reactive oxygen species detecting compounds according to embodiments of the present disclosure;

FIG. 3 depicts application of reactive oxygen species detecting compounds to a wound or body cavity according to embodiments of the present disclosure;

FIG. 4 depicts properties of two selected reactive oxidation species-sensitive chemiluminescent compounds;

FIG. 5 depicts a result of a study regarding in vivo imaging using a selected reactive oxidation species-sensitive chemiluminescent compound according to embodiments of the present disclosure;

FIG. 6A is a schematic illustration of a reactive oxygen species detecting nanoparticle according to an embodiment of the present disclosure;

FIG. 6B is a TEM image of reactive oxygen species detecting nanoparticles according to an embodiment of the present disclosure;

FIG. 7 is a chart that depicts chemiluminescence and fluorescence intensities at various H₂O₂ concentrations for reactive oxygen species detecting compounds according to embodiments of the present disclosure;

FIG. 8 a chart that depicts chemiluminescence and fluorescence intensity ratios at various H₂O₂ concentrations for reactive oxygen species detecting compounds according to embodiments of the present disclosure;

FIG. 9A a chart that depicts chemiluminescence and fluorescence intensities at various concentrations of reactive oxygen species detecting compounds according to embodiments of the present disclosure;

FIG. 9B a chart that depicts chemiluminescence and fluorescence intensity ratios at various concentrations of reactive oxygen species detecting compounds according to embodiments of the present disclosure;

FIG. 10 is a chart that depicts ratiometric spectra of reactive oxygen species detecting compounds reacted with selected free radical compounds according to embodiments of the present disclosure;

FIG. 11A depicts the effect of phantom depth on chemiluminescence and fluorescence intensity of a reactive oxygen species detecting compound according to embodiments of the present disclosure;

FIG. 11B is a chart depicting quantitative data reflecting the effect of phantom depth on chemiluminescence and fluorescence intensity and chemiluminescence/fluorescence intensity ratio of a reactive oxygen species detecting compound according to embodiments of the present disclosure;

FIG. 12 depicts bioluminescence and fluorescence imaging in mice with wound-induced inflammatory responses according to embodiments of the present disclosure; and

FIG. 13 depicts bioluminescence and fluorescence intensities in mice infected with selected quantities of bacteria according to embodiments of the present disclosure.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION

In the following description, reference is made to exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the spirit and scope of the present disclosure. The following detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, or characteristics may be combined in any suitable combinations and/or sub-combinations in one or more embodiments or examples.

Embodiments of the present disclosure provide methods, devices, components, and/or techniques for detecting reactive oxygen species (which may be referred to hereinafter as “ROS”), such as ROS released by polymorphonuclear neutrophils to indicate inflammation and/or infection. According to various embodiments, ratiometric ROS imaging may be carried out by using ROS-sensitive chemiluminescent compounds and ROS-insensitive fluorescent dye, as will be set forth in further detail below. Embodiments of the present disclosure include imaging probes for non-invasive detection, diagnosis, and/or monitoring of ROS activities. Such ROS activities may be associated with inflammatory responses, infection, and/or immune reactions; monitoring of ROS activities may provide intelligence of said inflammatory responses, infection, and/or immune reactions.

According to various embodiments of the present disclosure, ROS-sensing probe and coating designs are based on general principals of ratiometric imaging. As used in the present disclosure, the term “ratiometric imaging” includes, but is not limited to, observing an output by comparing the emission intensities of a combination of sources in response to an input. According to embodiments, ROS-sensing coatings include a ROS-sensitive chemiluminescent compound and a ROS-insensitive fluorescent dye that are covalently linked to sensors or devices via direct conjugation, polymer spacers, polymeric particles, and the like. As used herein, a ROS-insensitive dye may be referred to as a ROS-insensitive dye or a “reference dye.”

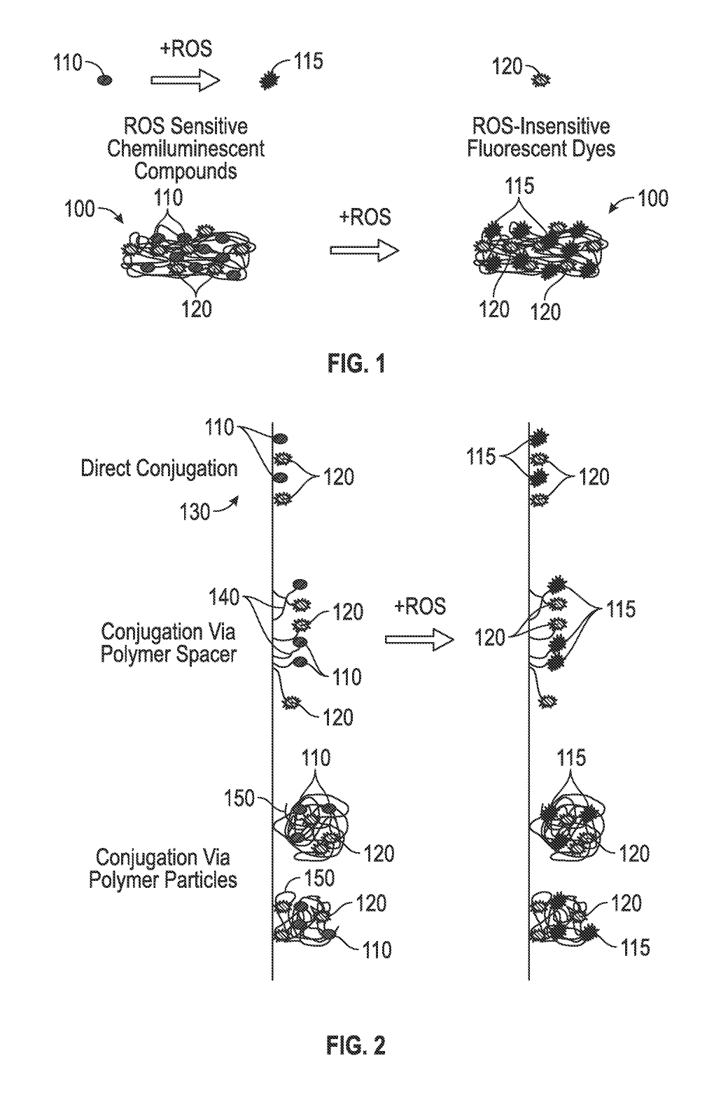

Referring to FIG. 1, a ROS-detecting compound 100 comprising a ROS-sensitive compound 110 and ROS-insensitive dye 120 is depicted. The ROS-sensitive compound 110 is adapted to spontaneously emit chemiluminescent signal 115 upon reaction with environmental ROS. ROS-insensitive fluorescent dye 120 is adapted to emit light when excited by light at particular wavelengths. However, according to some embodiments of the present disclosure, the intensity of emitted light from the ROS-insensitive fluorescent dye 120 is not influenced, or has a relatively small influence, by the presence of ROS. The ratio of observed signals from activated ROS-sensitive chemiluminescent compound 115 and fluorescent signals ROS-insensitive compound 120 can be analyzed to determine ROS intensity in situ in real time or near-real time.

Depending on the potential application and particular circumstances, various combinations of ROS-sensitive chemiluminescent dye 110 and ROS-insensitive fluorescent dye 120 with various emission wavelengths can be employed. Referring to FIG. 2, both groups (i.e., a compound selected from available ROS-sensitive chemiluminescent dyes 110 and a compound selected from available ROS-insensitive fluorescent dyes 120) of chemicals can be conjugated onto a device surface via direct conjugation 130, polymer spacers 140, polymeric particles 150, and the like. As would be understood by one of ordinary skill in the art having the benefit of this disclosure, a wide variety of ROS-sensitive compounds and ROS-insensitive dyes may be commercially available. Alternative embodiments of ROS-sensitive compounds 110 and/or ROS-insensitive dyes 120 include custom-made compounds to satisfy particular circumstances.

Various adaptable combinations of both compounds 110, 120 can be selected and used in applications according to embodiments of the present disclosure. In embodiments, ROS-insensitive fluorescent dyes 120 only emit light upon stimulation by light in an excitation wavelength spectrum corresponding to the dye 120. In some embodiments, the excitation and emission wavelengths of the ROS-insensitive dyes 120 are in visible and/or near-infrared light ranges, although near-infrared dyes may be most suitable for in vivo imaging. In embodiments, ROS-insensitive dyes 120 have various excitation and/or emission wavelengths. Examples of ROS-sensitive compounds 110 and ROS-insensitive dyes 120 are listed in Table 1 and Table 2, respectively.

TABLE 1
Examples of ROS-sensitive compounds.
L-012 (8-amino-5-chloro-7-phenylpyridol [3,4-d]
pyridazine-1,4 (2H,3H) dione)
Luminol (1,4-phthalazinedione, 5-amino-2,3-dihydro)
Lucigenin
MCLA (2-Methyl-6-(4-methoxyphenyl)-3,7-
dihydroimidazo[1,2-a]pyrazin-3-one Hydrochloride)
Green Chemiluminescent CD
Peroxalate nanoparticles
Peroxalate esters
Cypridina luciferin analogs
Pholasin

TABLE 2
Examples of ROS-insensitive dyes.
Acridine dyes
Cyanine dyes
Fluorine dyes
Oxazin dyes
Phenanthridine dyes
Rhodamine dyes
Cy-7
Dylight 800
IRDye ® 800
Alexa Fluor ® 790
HiLyte Fluor ™ 750
Oyster ® 800

Referring now to FIG. 2, according to various embodiments of the present disclosure, selected ROS-sensitive compounds 110 and ROS-insensitive dyes 120 are conjugated as part of nanodevices and/or on instrument or device surfaces via direct conjugation 130, polymer spacers 140, and/or polymer particles 150. In one embodiment, both compounds 110, 120 are placed on a device surface via encapsulation, absorption, adsorption, covalent linkage, and/or other association means that are known in the art or that will be known in the art. In an embodiment, both groups of compounds 110, 120 are conjugated to the surfaces via specific functional groups, such as carboxyl and amine groups. In embodiments, compounds 110, 120 are embodied as a spray, solution, wound dressing, surface coating, and/or medical implant.

The ratio of chemiluminescent intensity from activated ROS-sensitive compounds 115 in relation to fluorescent intensity from ROS-insensitive compounds 120 can be measured and analyzed to calculate the extent of ROS activities in the microenvironment near the ROS-sensitive compounds.

In embodiments, polymers are used as spacers 140 to place both ROS-sensitive dyes 110 and ROS-insensitive dyes 120 at the surfaces of devices and instruments. Suitable polymers include copolymers of water soluble polymers including, but not limited to, dextran, derivatives of poly-methacrylamide, PEG, maleic acid, malic acid, and maleic acid anhydride, and may include these polymers and a suitable coupling agent, including but not limited to 1-ethyl-3 (3-dimethylaminopropyl)-carbodiimide, also referred to as carbodiimide.

In some embodiments, polymers comprise degradable and/or nondegradable and/or polyelectrolyte material. Degradable polymer materials include, but are not limited to: poly-L-glycolic acid (PLGA), poly-DL-glycolic acid, poly-L-lactic acid (PLLA), PLLA-PLGA copolymers, poly-(DL-lactide)-block-methoxy polyethylene glycol, polycaprolacton, poly(caprolacton)-block-methoxy polyethylene glycol (PCL-MePEG), poly(DL-lactide-co-caprolactone)-block-methoxy polyethylene glycol (PDLLACL-MePEG), some polysaccharides (e.g., hyaluronic acid, alginate, polyglycan, N-acetylglucosamine, N-acetylgalactosamine, glucuronic acid, iduronic acid, chitosan, heparin/heparin sulfate, chondrotic/determatant sulfate), proteins (e.g., fibrinogen, albumin, collagen, complement, gelatin, extracellular matrix, immunoglobulins, fibronectin), peptides (e.g., RGD, polyhistidine), amino acid (e.g., L-arginine, histidine, lysine, aspartic acid, glutamic acid, serine, cysteine, valine, tyrosine, tryptophan), nucleic acids (e.g., RNA, DNA, single or double stranded), viruses, bacteria, cells and cell fragments, organic or carbon-containing materials, or combinations thereof.

Nondegradable materials include, but are not limited to: natural or synthetic polymeric materials (e.g., polystyrene, polypropylene, polyethylene teraphthalate, polyether urethane, polyvinyl chloride, silica, polydimethyl siloxane, acrylates, arcylamides, poly (vinylpyridine, polyacroleine, polyglutaraldehyde), some polysaccharides (e.g., hydroxypropyl cellulose, cellulose derivatives, Dextran®, dextrose, sucrose, Ficoll®, Percoll®, arabinogalactan, starch), and hydrogels (e.g., polyethylene glycol, ethylene vinyl acetate, N-isopropylacrylamide, polyamine, polyethyleneimine, poly-aluminum chloride), or combinations thereof.

In embodiments, ROS-sensitive compounds 110 and ROS-insensitive dyes 120 are linked to surfaces via particles 150 having micrometer or nanometer sizes. Particles 150 may be applied to the surface of the instrument and device by methods currently known in the art or methods that will be known in the art, including by physical adsorption or chemical conjugation. Certain techniques described in accordance with the present disclosure may be used in vivo and/or in vitro. For example, nanoparticles can be used for coating blood bags, blood tubes, and food process containers. Particles 150 are generally provided as a metal particle, carbon particle, inorganic chemical particle, organic chemical particle, ceramic particle, graphite particle, polymer particle, protein particle, peptide particle, DNA particle, RNA particle, bacteria/virus particle, hydrogel particle, liquid particle, porous particle, or combinations thereof. Thus, embodiments of particles 150 include, for example, metal, carbon, graphite, polymer, protein, peptide, DNA/RNA, microorganisms (bacteria and viruses), and polyelectrolyte, and may be loaded with a light or color absorbing dye, an isotope, a radioactive species, a tag, or have gas-filled pores. As used herein, the term “hydrogel” may refer to a solution of polymers, sometimes referred to as a sol, converted into gel state by small ions or polymers of the opposite charge or by chemical crosslinking. Various embodiments of the present disclosure include polymers listed above as polymer spacers 140.

According to some embodiments, a ROS-detecting compound 100 can be placed on surfaces of various types of devices and instruments. For examples, the ROS-detecting polymer coating 100 may be overlaid on top of medical devices. Such coatings 100 can provide ROS information at the interface between an implant and host tissues. In embodiments, the coating 100 is placed on the tip of a medical instrument to allow detection of ROS changes in situ. Changes of ROS have been shown to affect protein structure and activities; thus, in embodiments, the coating 100 is placed in a storage container to detect the ROS activities of materials in the container. In other embodiments, the coating 100 is placed in or on food processing equipment to monitor chemistry and reactions of ROS in foods.

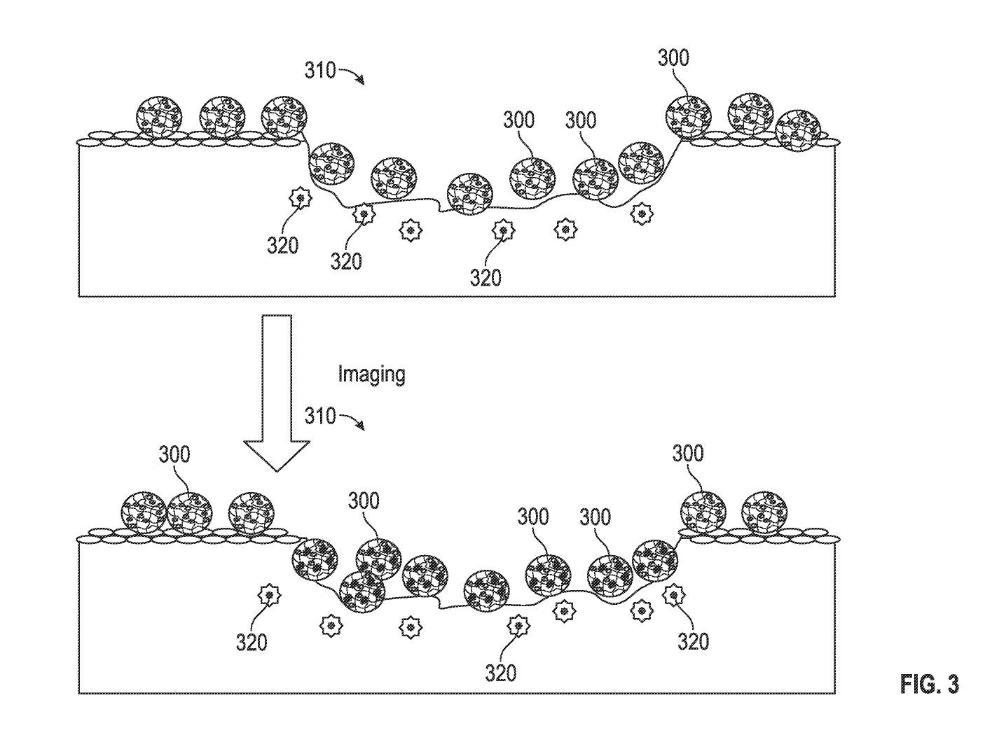

Referring now to FIG. 3, embodiments of the present disclosure provide ROS sensing probes 300 comprising a ROS-detecting compound 100. In operation, probes 300 can be placed on top of and/or at a wound 310, tissue surface, and/or body cavity to detect the extent of ROS 320 activity therein or thereat. As will be understood by those of ordinary skill in the art having the benefit of this disclosure, severe inflammatory responses and infection may lead to the production of ROS 320. By measuring ROS 320 activities in the wound 310, a healthcare provider can monitor the healing status of the wound 310 and detect potential complications (such as infection) relatively quickly.

According to various embodiments of the present disclosure, after application of ROS-detecting compound 100, the subject target may be imaged after a waiting time. In embodiments, the waiting time may range from one minute to two hours. Other wait times may selected according to particular circumstances.

In embodiments, probes 300 can be placed on a device surface in concentrations ranging from 50 ng to 1 mg per centimeter square. The concentrations of probes 300 can be placed on and/or at a wound 310, tissue surface, and/or body cavity in concentrations ranging from 1 microgram/milliliter to 1 milligram/milliliter.

The ratio of ROS-sensitive chemiluminescent intensity 115 and ROS-insensitive fluorescent intensity 120 can be measured and analyzed to determine the extent of ROS 320 activities in the wound 310, tissue surface, and/or body cavity. In one embodiment, the wound 310, tissue surface, and/or body cavity is imaged with no filter to detect the intensities of ROS-sensitive chemiluminescence 115 and imaged with a specific wavelength filter for ROS-insensitive fluorescence 120. The specific wavelength filter corresponds to an emission wavelength of the ROS-insensitive fluorescent dye 120. In embodiments, the ROS-insensitive fluorescent dye 120 is excited during imaging by applying specific wavelength light onto the dye 120. Ratiometric chemiluminescent imaging can thus be used to quantitatively detect production of ROS in inflammatory and/or infected tissues.

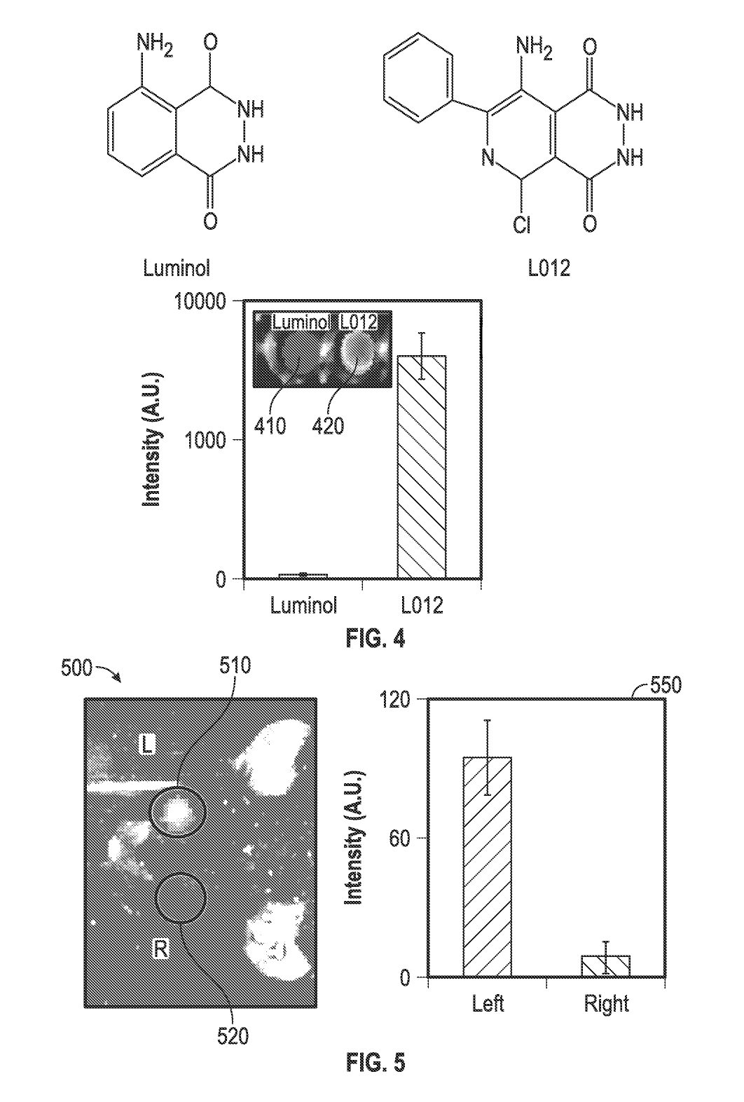

Referring to FIG. 4, chemical structures for luminol (1,4-phthalazinedione, 5-amino-2,3-dihydro) and L-012 (8-amino-5-chloro-7-phenylpyridol[3,4-d]pyridazine-1,4 (2H,3H)dione) are depicted. To demonstrate the effectiveness of ROS probes, luminol and L-012 were added to 250 μl reaction mixtures containing 1×10⁶ PMNs and 2 mM L-012 or 2 mM luminol. The ROS measurements were initiated by adding 10 μl of PMA (6.5 nM) at room temperature. Chemiluminescence images 410, 420 were captured with a 5 minute acquisition time using a Kodak In Vivo FX Pro system (f/stop, 2.5; no optical filter, 4×4 binning).

To compare the ROS detection sensitivity of L-012 vs. luminol, mouse peritoneal PMNs were incubated with L-012 in the presence of the protein kinase C activator PMA. With PMA activation, both luminol and L-012 emitted chemiluminescence. However, it was observed that the signal intensity generated by the L-012 was approximately 1000-fold greater than that generated by luminal.

Referring now to FIG. 5, subsequent studies were carried out to evaluate the feasibility of L-012 for in vivo oxidative stress imaging. L-012 (10 μL, 15 mg/mL) was topically dropped on a mouse's eyes with hypertension-suffering left eye 510 or control right eye 520. The animal was sedated and then imaged with a Kodak in vivo FX imaging system, resulting in image 500. A strong bioluminescence signal was found in the hypertension-suffering left eye 510, while control right eye 520 emitted low or no chemiluminescence. Quantification analysis 550 showed that with regard to the control eyes, there was a ten-fold signal enhancement in the hypertension-suffering eye 510.

As will be understood by one of ordinary skill in the art having the benefit of this disclosure, ratiometric measurements have an advantage in that factors such as fluctuation of the excitation source and sensor concentration may not affect the signal intensity ratio between the indicator 115 and reference dye 120.

In embodiments of the present disclosure, a ratiometric chemiluminescence nanoprobe 600 comprises a core-shell polymeric nanoparticle encapsulation 610 having a chemiluminescence agent 110 and a reference near infra-red dye 120, as described in further detail below. In one embodiment, the chemiluminescence agent 110 comprises L-012 and the reference near infra-red dye 120 comprises 1,1′,3,3,3′,3′-Hexamethylindotricarbocyanine iodide (abbreviated herein as “IR750”).

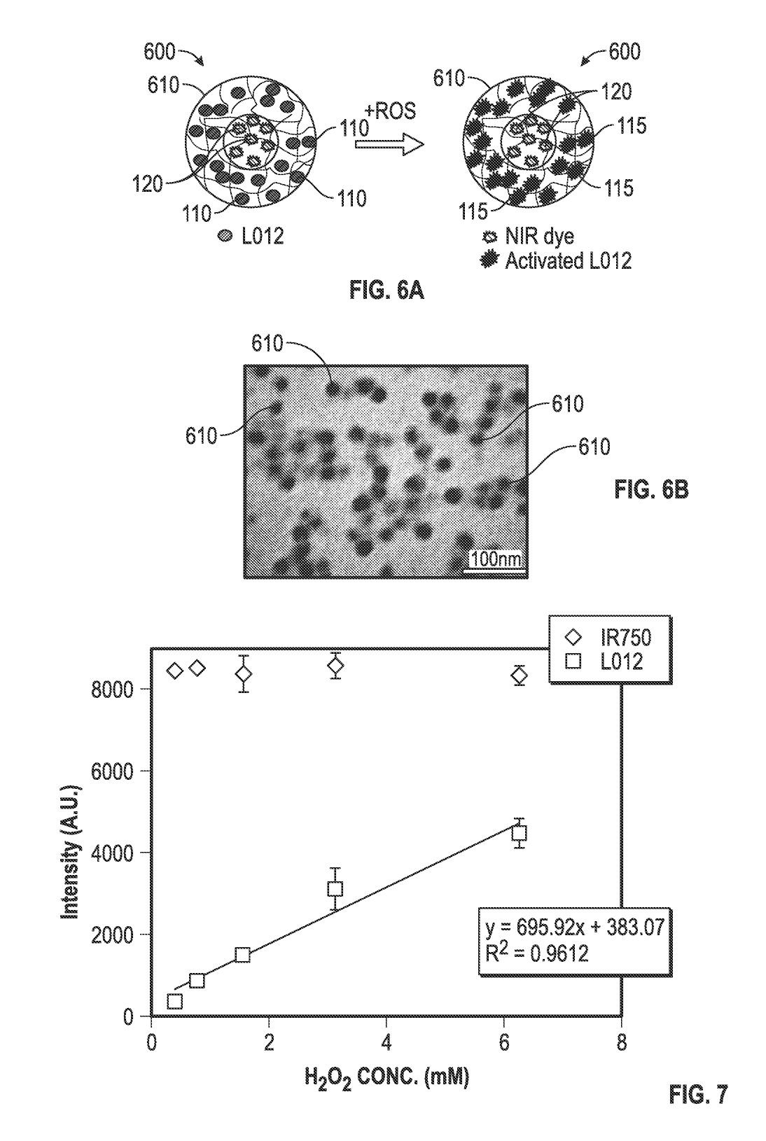

Referring to FIG. 6A, according to embodiments, the nanoprobe 600 may be manufactured by the following procedure. First, the core-shell nanoparticle 610 as the carrier may be prepared by emulsion polymerization. 67 mM 2-(2-methoxyethoxy) ethyl methacrylate (MEO2MA), 34 mM poly(ethylene glycol) methyl ether methacrylate (MEO4MA), 34 mM ethylene glycol dimethacrylate (EGDMA), 34 mM styrene (St), and 4 mM sodium dodecyl sulfate (SDS) are all added in distilled water in a three-neck flask, and the flask is placed in a circulating bath of water at approximately 70° C. under N₂ gas for 30 minutes. Polymerization is initiated by adding 2 mM ammonium persulfate (APS), and the reactions are carried out at 70° C. for 6 hours under N₂ gas. The resultant PEG-co-St nanoparticles (NPs) 610 may be purified with dialysis against deionized water for 1 week and then stored at 4° C. for subsequent use.

Referring to FIG. 6B, TEM images of the PEG-co-St NPs revealed homogeneous and spherical morphology with the core-shell nanoparticles 610 having an average diameter of approximately 25 nm.

Next, to prepare ratiometric ROS probes, the reference dye 120 and L-012 110 may be sequentially physically encapsulated into the PEG-co-St NPs 610 as follows: First, 3 mg IR750 is dissolved in 5 ml tetrahydrofuran (THF) to prepare the dye 120 solution. The dye 120 solution is then added to 10 ml PEG-co-St NP 610 dispersion (2.0 mg/ml), and the mixture incubated overnight in a dark environment. In some embodiments, THF solvent is removed under vacuum. The IR750-trapped NPs is then purified with dialysis against DI water. The loading efficiency of IR750 dye 120 may be estimated to be 1.2 wt % according to the following equation:

efficiency=(M_{loadedIR750}/M_{NP})×100%

Where:

M_{loadedIR750} is the mass of the entrapped dye 120 and

M_{NP} is mass of the NPs.

Given its hydrophobic property, IR750 may be physically encapsulated into the polystyrene cores of the NPs by the hydrophobic association. As the hydrophobic microenvironment of polystyrenes may be virtually impermeable to water and oxidative agents such as ROS species, the photo stability of the physically-entrapped IR750 120 may be significantly enhanced.

Further, ROS probe L-012 110 may then be loaded into PEG network (shell) of the IR750-NP to prepare the ROS ratiometric probe 600. In brief, 50 mg L-012 can be added to 10 ml IR-NP aqueous dispersion (2.0 mg/ml). After the L-012 110 is dissolved under stirring, the mixture may be incubated overnight in a dark environment. The unloaded L-012 can be removed by centrifugal filter. The filtrate may be collected and L-012 amount in the filtrate determined by absorbance at 455 nm using UV-visible spectrometer. The loading efficiency may be estimated to be 45 wt % according to the following equation:

efficiency=(M_{total L012}−M_{filtrate L012})/M_{NP}×100%

Where:

M_{total L-012} is the mass of the total added L-012;

M_{filtrate L-012} is the mass of the L-012 in filtrate; and

M_{NP} is mass of the NPs.

Referring now to FIG. 7, testing of nanoprobe 600 (abbreviated herein as “IR750-NP-L-012”) for ROS detection in vitro was carried out in H₂O₂ concentration ranging from 0.04 to 0.625 mM. The chemiluminescence and fluorescence intensities were recorded with a microplate reader (Infinite M200; Tecan, Männedorf, Switzerland) with 2 minutes and 20 μs acquisition time, respectively. It was found that with increases in H₂O₂ concentration, the average chemiluminescence intensities were linearly increased while the average fluorescence intensities (excitation at 760 nm, emission at 830 nm) were unchanged. The ratios of average intensities between L-012 (chemiluminescence) and IR750 (fluorescence) can be calculated to provide quantitative measurement of H₂O₂ in the aqueous environment.

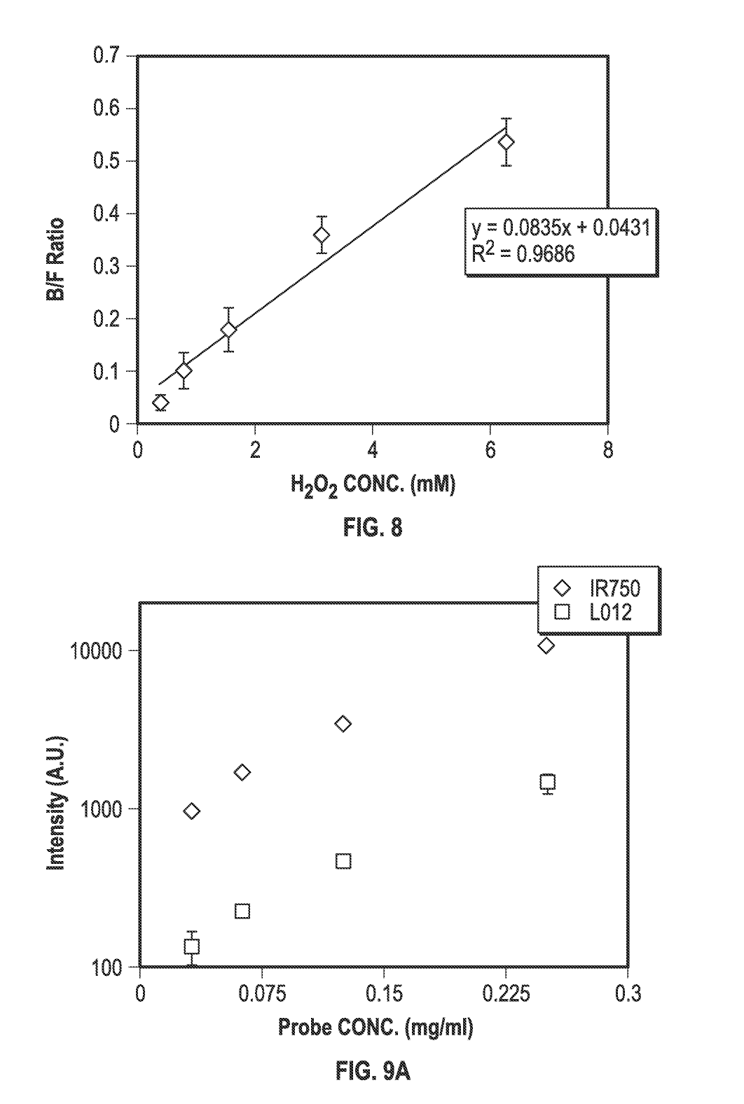

Referring now to FIG. 8, light intensity ratio of chemiluminescence to fluorescence at different H₂O₂ concentrations are demonstrated. It may be observed that the intensity ratios display a strong correlation with H₂O₂ concentration from 0.04 to 0.625 mM (Ratio=0.8347×H₂O₂+0.0431 and R²=0.97).

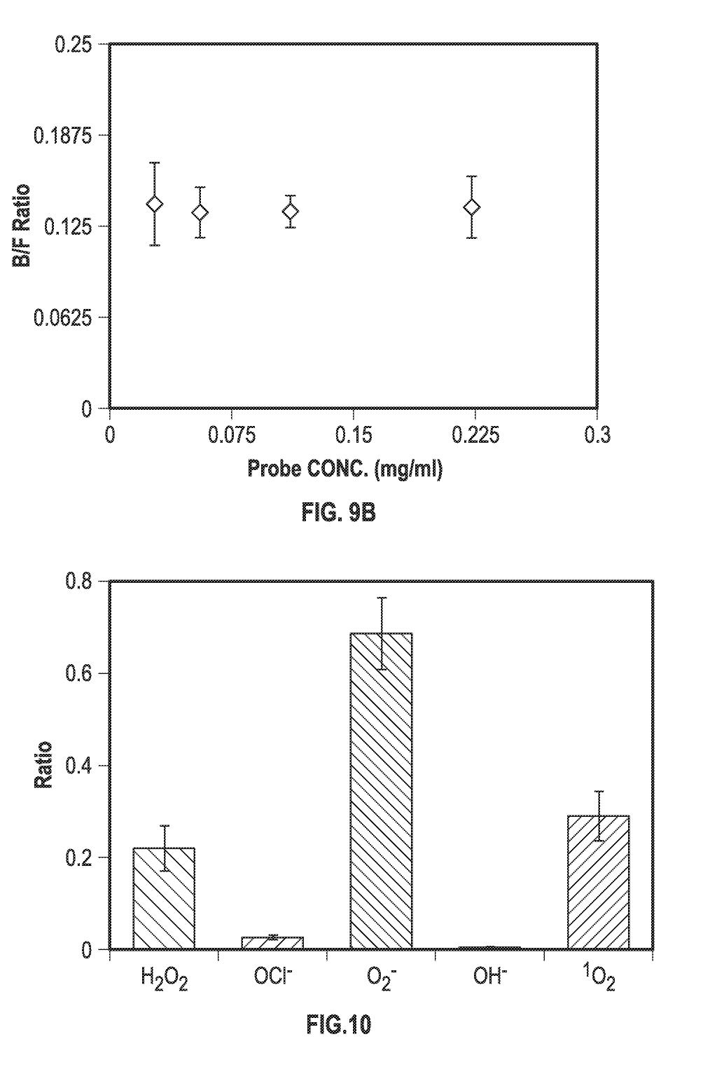

Referring now to FIGS. 9A and 9B, the effect of the probe concentration on chemiluminescence/fluorescence intensities and their ratios are demonstrated. FIG. 9A depicts chemiluminescence and fluorescence intensities with different probe concentrations. FIG. 9B depicts the intensity ratio of chemiluminescence to fluorescence at different probe concentrations. It may be observed that as the probe concentration is increased from 0.03 mg/ml to 0.25 mg/ml, the intensities for L-012 (chemiluminescence) and IR750 (fluorescence) are amplified. However, the intensity ratios of both L-012 and IR750 are nearly constant at about 0.14-fold, independent of the sensor concentrations. As would be understood by one of ordinary skill in the art having the benefit of this disclosure, these results demonstrate that the synthesized ratiometric chemiluminescence IR750-NP-L-012 nanoprobe 600 can be used to in vitro determine H₂O₂ concentrations using ratiometric imaging techniques.

Referring now to FIG. 10, the sensitivity of IR750-NP-L-012 to other ROS species has been investigated. For example, the IR750-NP-L-012 nanoprobes 600 were exposed to various radical and nonradical reactive species such as superoxide anion (O₂—), hydrogen peroxide (H₂O₂), singlet oxygen (¹O₂), and hypochlorite anion (OCl—). The reactions took place in PBS buffer using the same nanoparticle concentration, reaction volume, and reaction time for each. The intensity ratios between L-012 (chemiluminescence) and IR750 (fluorescence, emission at 830 nm) was collected using a microplate reader (Infinite M200; Tecan, Männedorf, Switzerland). As shown in FIG. 10, ratiometric ratio spectra of IR750-NP-L-012 nanoprobes 600 reacted with various types of free radicals, including H₂O₂, OCl—, O₂—, OH—, ¹O₂. The observed intensity ratio was 0.22, 0.03, 0.69, 0.005, and 0.29 for H₂O₂, OCl—, O₂—, OH—, and ¹O₂ radicals, respectively. These results suggest that the IR750-NP-L-012 may be effectively used to detect various reactive oxygen species.

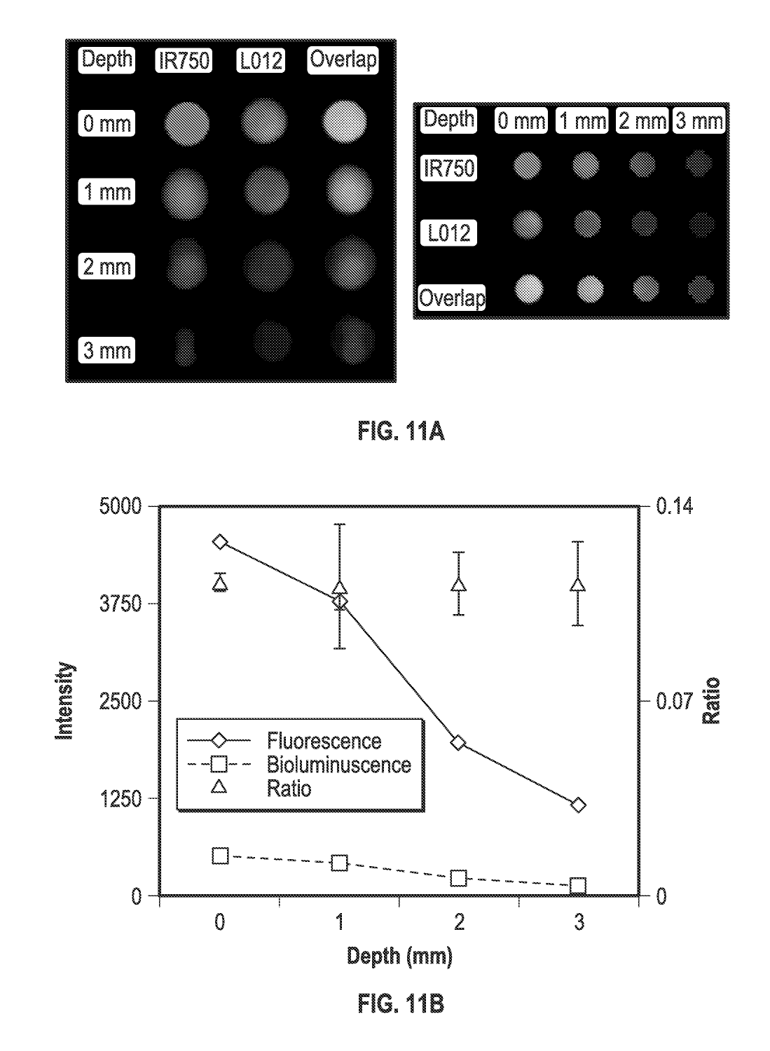

Referring now to FIGS. 11A and 11B, the effect of tissue depth on the intensity ratio of L-012 (chemiluminescence) and IR750 (fluorescence) was investigated. Skin phantom with varying thicknesses (1-3 mm) were prepared according to the reference displayed. These phantoms were placed between the IR750-NP-L-012 probes and the light source during image collection. Specifically, the phantoms were placed on well bottoms in a 96-well plate. 250 μl of H₂O₂ and 15 μl of IR750-NP-L-012 probes (05 mg/ml) were added into the wells. Using a Kodak In Vivo FX Pro System (Kodak, USA) (f/stop, 2.5; 4×4 binning), the chemiluminescence images were taken with an exposure time of 5 minutes, and the fluorescence images were taken with an exposure time of 2 seconds (while exciting the samples at 760 nm and observing emission at 830 nm). The mean chemiluminescence and fluorescence intensities for all pixels in the images were calculated using Carestream Molecular Imaging Software, Network Edition 4.5 (Carestream Health). The chemiluminescence and fluorescence intensities decreased with increasing skin-phantom thickness. Nevertheless, the intensity ratios of both chemiluminescence and fluorescent intensities were nearly constant independent of skin-phantom thickness (the L-012/IR750 ratio was approximately 0.56).

Recruited activated PMNs activate the respiratory burst, releasing a variety of reactive oxygen species, including superoxide, hydrogen peroxide, and hyperchlorous acid. It was investigated whether the IR750-NP-L-012/probes 600 were able to detect ROS generated by isolated neutrophils (PMNs). PMN may be obtained via isolation from the peritoneal cavity after casein stimulation. A number of PMN (5×10⁴ to 1×10⁶) were incubated in 0.25 ml of 0.9% NaCl solution containing 10 mM phosphate buffer (pH 7.4), 6 mM KCl, and 6 mM MgCl₂ in the presence of the nanoprobes (10 μl, 0.5 mg/ml). After incubation of the mixture for 3 min at 37° C., the reaction were initiated by adding 6 nM phorbol 12-myristate 13-acetate (PMA). During the incubation, chemiluminescence and fluorescence intensities were recorded continuously for 60 minutes using a microplate reader. Activated PMNs prompted a strong signal of chemiluminescence and chemiluminescence intensities that increased with cell number increase; however the fluorescence intensity of IR750 was independent of cell count. In addition, a positive linear relationship was observed between the intensity ratio and the activated PMN number (R²=0.95).

To verify whether the chemiluminescence signals resulted from ROS generated by activated PMNs, similar PMN-mediated chemiluminescence measurements were carried out in the presence or absence of Tempol, a superoxide dismutase mimetic/ROS neutralizer. The chemiluminescence intensity was substantially reduced in the presence of as little as 5 mM Tempol while fluorescence signal remained constant. Collectively, these data indicate that the IR750-NP-L-012 probes 600 can be used to estimate the numbers of activated PMN based on their ROS-mediated intensity ratio. These results support the notion that the IR750-NP-L-012 probes 600 may be used to quantify the extent of PMN-associated ROS production.

The cytotoxicity of the IR750-NP-L-012 probes 600 was determined using 3T3 fibroblasts and MTT assay. It was observed that the IR750-NP-L-012 probes 600 trigger no statistically significant cytotoxicity (i.e., less than 15% cell death) over the studied concentration range (up to 0.8 mg/ml), indicating that the above-prepared the IR750-NP-L-012 probes 600 possess adequate cell compatibility for further in vivo testing.

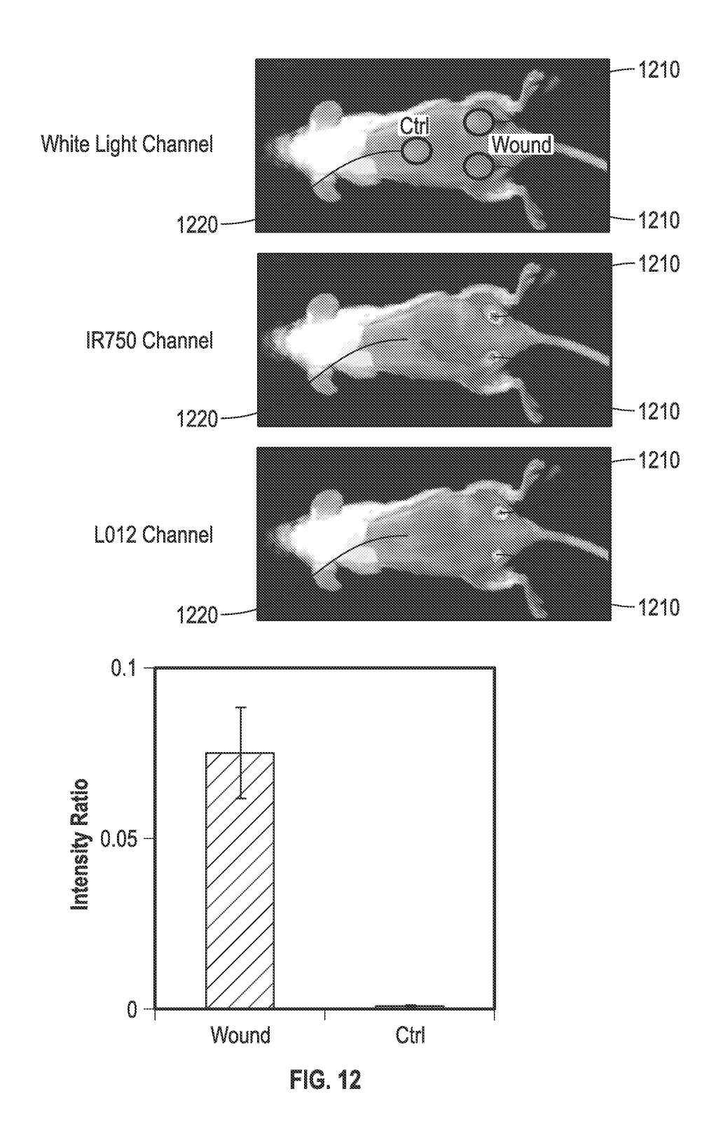

To assess whether the IR750-NP-L-012 probes 600 can be used to assess ROS production arising in vivo, an animal wound model had been applied. Referring to FIG. 12, a topical wound 1210 with diameter of 8 mm was created on the back of mice (n=4), and non-wounded skin 1220 was considered as a control. 24 hrs after creating the wound 1210, 100 of the IR750-NP-L-012 probes 600 (0.5 mg/ml) were administered topically on the wound 1210. Using a Kodak In Vivo FX Pro System (Kodak, USA) (f/stop, 2.5; 8×8 binning), L-012 signal was collected at an exposure time of 10 minutes, and fluorescence signal was recorded at an exposure time of 2 seconds with the excitation at 760 nm and observing emissions at 830 nm. Results showed a strong signal at the wound 1210 relative to control sites 1220. However, for chemiluminescence channel, the signal at the control 1220 was not detected while there was a strong signal at the wound site 1210. Quantitative analysis suggested that the intensity ratio (chemiluminescence/fluorescence) was 70 times higher at the wound site 1210 compared with the control site 1220. These results reveal that the IR750-NP-L-012 probe 600 can be used to noninvasively monitor real-time inflammation-induced ROS generation in vivo.

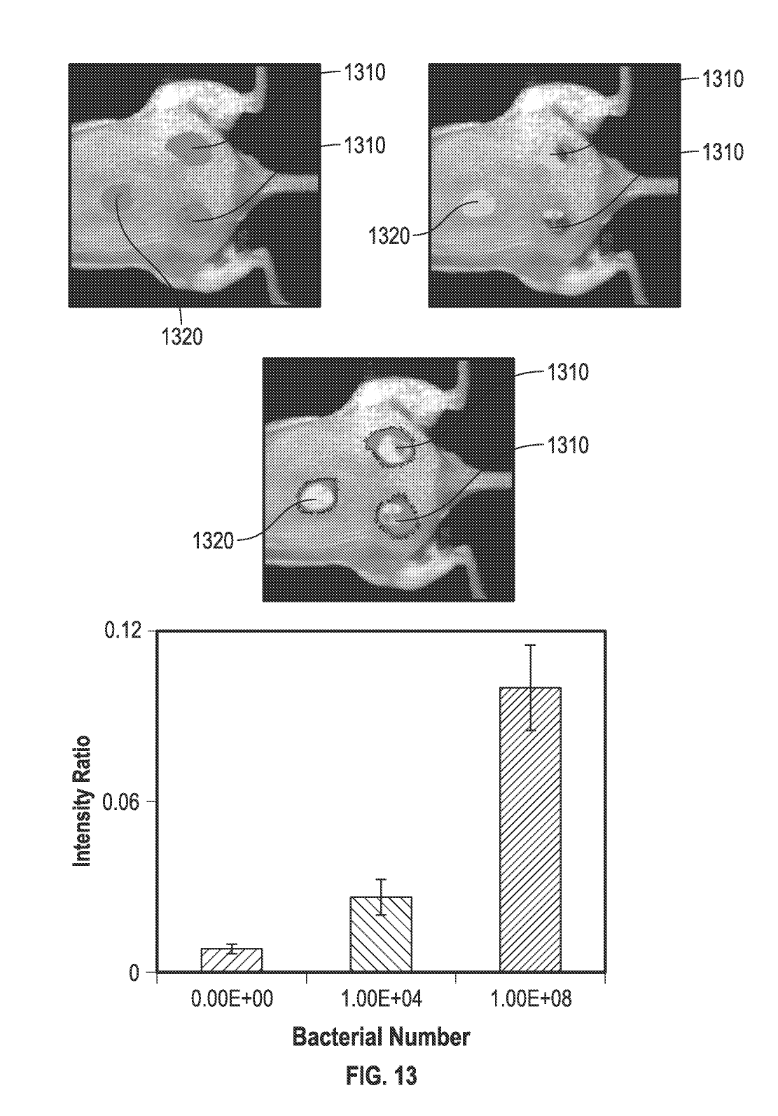

It was investigated whether the IR750-NP-L-012 probe 600 can be used to monitor and diagnose ROS activities in an infected wound using a subcutaneous infected wound mouse model. Referring now to FIG. 13, to simulate infected wound, biofilm-forming strain RN6930 of Staphylococcus aureus was chosen for this study. The bacteria were cultured at 37° C. and maintained in brain heart infusion broth for the duration of this study. After washing thrice with sterile PBS, the bacterial solution was diluted to 1×10⁵ and 1×10⁹ per ml with PBS prior to the experiment. The animal study was carried out using Balb/c mouse with 4 animals per group. Mouse was anesthetized with isoflurane inhalation during the implantation procedure. Immediately following sedation, the dorsal skin of test animals were shaved, sterilized with 70% ethanol and then subcutaneously injected with 0.1 cc of bacterial solution (1×10⁴ and 1×10⁸ per wound) (for inducing infected wound 1310). The contra-lateral upper back 1320 of the same animal was injected with 0.1 cc of normal saline and served as the control. One day after treatments, the wound 1310 and surrounding tissues were administered topically with the IR750-NP-L-012 probe 600. The injection cavity was assessed with the Kodak Imaging system to determine the ROS response to the injected material in the subcutaneous tissue. Following the imaging, a thin slice of infected tissue was isolated for histological evaluation and microscopic examination. As shown in FIG. 13, drastic changes in ROS ratiometric imaging in vivo that strongly depended on the incubated bacterial number were observed. The ratiometric ratios were calculated to compare with control 1320 (no bacteria). The control site 1320 exhibited minimal or no significant ratiometric changes, while the more implanted bacteria triggered exhibited more ratiometric change. Quantitative analysis showed that there was a near-linear relationship between the intensity ratio and the implanted bacterial number. Histological analysis suggested that the implanted bacteria recruited more inflammatory cell accumulation. A positive linear relationship can be observed between inflammatory cell and intensity ratio. These data indicated that the extent of the inflammatory response associated with the infection may be assessed by the ROS ratiometric imaging measurement in real time and in vivo.

Although the present disclosure is described in terms of certain preferred embodiments, other embodiments will be apparent to those of ordinary skill in the art, given the benefit of this disclosure, including embodiments that do not provide all of the benefits and features set forth herein, which are also within the scope of this disclosure. It is to be understood that other embodiments may be utilized, without departing from the spirit and scope of the present disclosure.

1. A composition for detecting reactive oxidation species activities comprising:
a reactive oxidation species-sensitive chemiluminescent compound and
a reactive oxidation species-insensitive fluorescent reference dye;
wherein the reactive oxidation species activities indicate an inflammatory response, an infection, or an immune reaction.

2. The composition of claim 1, wherein the chemiluminescent compound and the fluorescent reference dye are covalently linked via direct conjugation.

3. The composition of claim 1, wherein the chemiluminescent compound and the fluorescent reference dye are covalently linked via polymer spacers.

4. The composition of claim 1, wherein the chemiluminescent compound and the fluorescent reference dye are covalently linked via polymeric particles.

5. The composition of claim 1, wherein the chemiluminescent compound and the fluorescent reference dye are covalently linked to a surface.

6. The composition of claim 1, wherein the chemiluminescent compound and the fluorescent reference dye are encapsulated in a nanoprobe.

7. The composition of claim 1 in the form of a coating.

8. The composition of claim 1, wherein the chemiluminescent compound is selected from the group consisting of L-012, luminol, MCLA, green chemiluminescent CD, peroxalate nanoparticles, a peroxalate ester, an analog of Cypridina luciferin, and pholasin.

9. The composition of claim 1, wherein the fluorescent reference dye comprises a water-insoluble fluorescent cyanine dye.

10. The composition of claim 1, wherein the composition comprises one selected from the group consisting of a spray, a solution, a wound dressing, a surface coating, a medical implant, and an instrument.

11. (canceled)

12. (canceled)

13. (canceled)

14. (canceled)

15. (canceled)

16. (canceled)

17. (canceled)

18. A compound for detecting reactive oxidation species activities comprising a nanoparticle encapsulating:
a reactive oxidation species-sensitive chemiluminescent compound and
a reactive oxidation species-insensitive fluorescent reference dye;
wherein the reactive oxidation species activities indicate an inflammatory response, an infection, or an immune reaction.

19. The compound of claim 18, wherein the nanoparticle comprises a polymeric nanoprobe.

20. The compound of claim 18, wherein the chemiluminescent compound is selected from the group consisting of L-012, luminol, MCLA, green chemiluminescent CD, peroxalate nanoparticles, a peroxalate ester, an analog of Cypridina luciferin, and pholasin.
